(19) United States
(12) Patent Application Publication
(10) Pub. No.: US 2005/0173472 A1
(43) Pub. Date: Aug. 11, 2005

(54) Angled caulk tube extension

(76) Inventor: Steven M. Page, Mokena, IL (US)

Correspondence Address: Mark E. Wiemelt, 10 S. LaSalle St., Ste. 3300, Chicago, IL, 60603, US

(21) Appl. No.: 10/776,904

(22) Filed: Feb. 11, 2004

(51) Int. Cl. A61F 2/30

(52) U.S. Cl. 222/568; 222/327

(57) ABSTRACT

The present invention relates to a removable, angled caulk tube extension dimensioned for coupling with a standard caulk canister spout. The caulk tube extension is an elongated cylindrical tube containing an open attaching end and an open dispensing end and containing a first segment and a second segment, with the longitudinal axis of each segment oriented at an angle to one another. The cylindrical tube tapers as it extends from the attaching end and is at its narrowest diameter at the dispensing end. The caulk tube extension is manufactured from a substantially rigid material, such as a relatively high density polyethylene, and contains male threads disposed circumferentially about the interior surface of the open attaching end. The caulk tube extension is manufactured from a harder material than is the caulk canister spout so that the caulk canister spout is self-threaded when the caulk tube extension is mated thereto. Once attached, caulking compound is directionally dispensed through the open dispensing end in accordance with the angle of the tube.

The present invention is the subject of Disclosure Document No. 506214 filed on Feb. 13, 2002.

### BACKGROUND OF THE INVENTION

1. Field of the Invention

The present invention relates to improvements of the application of sealers. More particularly, the present invention relates to nozzles for dispensing caulking compounds and similar viscous materials and specifically to an angled caulk tube extension which functions to allow the directional dispensing of the caulking compound.

2. The Prior Art

Caulking is typically used to provide a resilient seal between two adjacent structures, such as a window frame and a brick wall, to prevent water and/or air from entering the gap between the two adjacent structures. Although the installation of caulking is desirable, it is often difficult to dispense the viscous caulk into the gap between the structures with the straight, tapered caulk tube conventionally provided with the caulk container. The application of caulking compound to corners, deep cracks and crevices poses a particular challenge to conventional caulk tube nozzles. Often the space to be caulked is oriented in such a way that the main body of the caulking gun necessarily makes contact with surrounding surfaces during application, thereby interfering with or altogether preventing proper application of the caulking compound. It would be beneficial, therefore, to have a caulk tube nozzle that was removably attachable over the caulk tube of the caulk container and which was positioned at an angle favorable to the application of caulking compound into such gaps. Furthermore, it would be beneficial to have a nozzle that was sufficiently rigid so as to more easily enable proper application of the caulking compound into difficult locations such as these, and to do so in a manner that resulted in a smooth, continuous flow with no discernible discontinuities.

The use of caulking nozzles is known in the prior art. More specifically, caulking nozzles heretofore devised and utilized for the purpose of distributing filler material are known to consist basically of familiar, expected and obvious structural configurations which are suited for the specific individual purposes they address, but differ from the present invention.

### SUMMARY OF THE INVENTION

The present invention advantageously overcomes the limitations in the conventional art and prior solutions. In view of the disadvantages inherent in the known types of caulking nozzles present in the prior art, the present invention provides an improved caulking nozzle for applying caulking compounds in hard to reach areas such as corners, deep cracks and crevices, which pose a particular challenge to conventional caulk tube nozzles. Often the space to be caulked is oriented in such a way that the main body of the caulking gun necessarily makes contact with surrounding surfaces during application, thereby interfering with or altogether preventing proper application of the caulking compound.

To facilitate the application of caulk to such areas, the present invention consists of a rigid, elongated, angled tube having an open attaching end and open dispensing end. The open attaching end is dimensioned for coupling with a standard caulk tube and receives caulking compound therefrom. After positioning the angled tube onto the caulk tube, caulking compound is directionally dispensed through the open dispensing end in accordance with the angle of the tube. Because the angled tube is rigid, the angle of attack is more easily maintained throughout application of the caulking compound. The result is a smooth, continuous bead with virtually no cosmetically discernible discontinuities, a result that is difficult to achieve with prior art devices.

There has thus been outlined, rather broadly, the more important features of the present invention so that the detailed description of the preferred embodiment that follows may be better understood, and so that the present contribution to the art may be better appreciated. There are additional features of the present invention that will be described hereinafter in the detailed description of the preferred embodiment and which will form the subject matter of the claims appended hereto.

In this respect, before explaining at least one embodiment of the present invention in detail, it is to be understood that the present invention is not limited in its application to the details of construction and to the arrangements of the components set forth in the following description or illustrated in the drawings. The present invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the present invention in any way.

These together with other objects of the present invention, along with the various features of novelty which characterize the present invention, are pointed out with particularity in the claims annexed to and forming a part of this disclosure. For a better understanding of the present invention, its operating advantages and the specific objects attained by its uses, reference should be had to the accompanying drawings and descriptive matter in which there is illustrated a preferred embodiment of the present invention.

It is therefore an object of the present invention to provide a caulk tube extension that is angled, rigid and facilitates the flow of caulk into hard to reach areas.

It is another object of the present invention to provide a caulk tube extension that is economical to manufacture, convenient to use, and results in a savings of time and expense, while at the same time providing a superior result than is achieved with prior art devices.

It is a further object of the present invention to provide an improved extension easily adapted and attachable to standard containers of caulking compound.

It is a further object of the present invention to provide an angled, rigid nozzle that can stand up to the more vigorous application methods sometimes required to force caulking compound into tight areas.

An advantage of the present invention is the rigidity of the tip which allows the operator to easily maintain the proper angle of attack throughout the application process.

Another advantage of the present invention is that the rigid nozzle enables a smooth, continuous bead to be applied with virtually no cosmetically discernible discontinuities.

These and other objects and advantages of the present invention will become apparent in view of the present specification, claims and drawings.

### BRIEF DESCRIPTION OF THE DRAWINGS

The foregoing and other additional objects of the present invention will be readily appreciated by those skilled in the art upon gaining an understanding of the preferred embodiment as described in the following detailed description and shown in the accompanying drawings in which:

FIG. 1 is a cross sectional side view of the caulk tube extension.

FIG. 2 is a bottom view of the caulk tube extension showing the attaching end.

FIG. 3 is a top view of the caulk tube extension showing the dispensing end.

FIG. 4 is an enlarged perspective view showing the exterior of the caulk tube extension.

FIG. 5 is a schematic perspective showing the caulk tube extension attached to the nozzle of a caulk canister.

FIG. 6 is a side view of the caulk tube extension shown positioned with respect to a caulking gun.

### DETAILED DESCRIPTION OF THE PREFERRED EMBODIMENT

While this present invention is susceptible of embodiments in many different forms, there are shown in the drawings and will be described in detail herein, a preferred embodiment, with like parts designated by like reference numerals and with the understanding that the present disclosure is to be considered as an exemplification of the principles of the present invention, and is not intended to limit the claims to the illustrated preferred embodiment.

FIG. 1 shows the features of the angled caulk tube extension 8 in a cross sectional side view. The open attaching end 13 contains an interior surface 31 covered by a plurality of male threads 32. The diameter of the open attaching end 13 is sized to fit over the spout 20 of a conventional caulking canister 40. The exterior surface 33 contains a collar 12 adjacent to which is an integrally connected hex nut 14. The first segment of the tube 10 tapers as it extends from the open attaching end 13. Approximately two inches from the hex nut 14, the axis angles at approximately 45° from the longitudinal axis of the first segment of the tube 10. The second segment of the tube 11 extends therefrom and continues tapering until the open dispensing end 18 is reached approximately 3.75 inches later. The diameter of the open dispensing end 18 is preferably 0.125 inches. In its preferred embodiment, the angled caulk tube extension 8 is made of a relatively high density polyethylene, although other relatively rigid materials selected from the group consisting of plastic, plastic composites, epoxy and other similar materials will suffice. The preferred method of manufacturing is plastic injection molding.

FIG. 1 also illustrates the position of the longitudinal axis of the first segment of tube 10 relative to that of the second segment of tube 11. The angle created thereby is advantageous to the application of caulking compound in tight, hard to reach areas. Furthermore, the rigidity of the caulk tube extension 8 lends itself to the application of the caulking compound in a smooth, continuous bead with virtually no visibly discernible discontinuities.

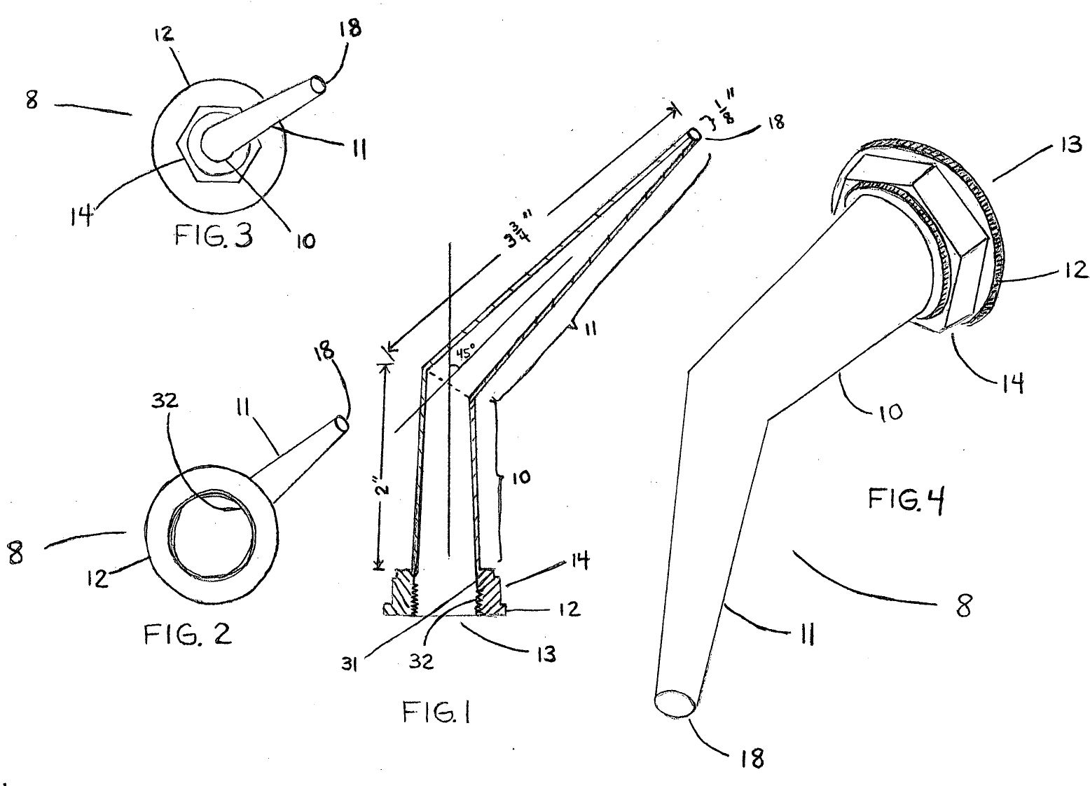

FIG. 2 shows a bottom view of the open attaching end 13 and illustrates the male threads 32 disposed circumferentially about the interior surface 31. Also displayed is the orientation of the second segment of the tube 11 and the open dispensing end 18 relative to the open attaching end 13.

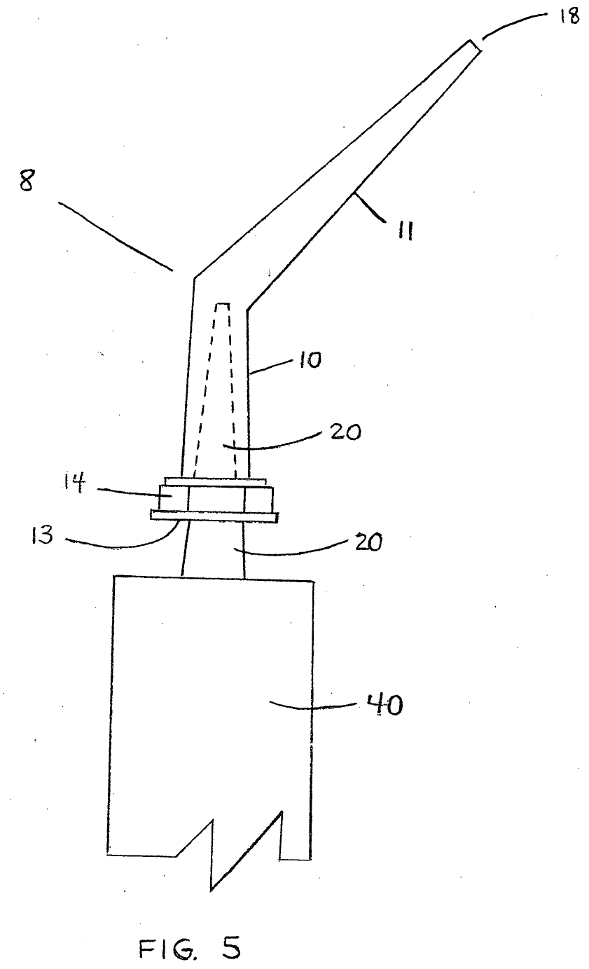

FIG. 3 shows a top view of the open attaching end 13 and illustrates the integrally connected hex nut 14 used to tighten the caulk tube extension 8 onto the caulk canister spout 20. Also displayed is the orientation of the second segment of the tube 11 and the open dispensing end 18 relative to the open attaching end 13.

FIG. 4 shows an enlarged perspective view showing the exterior of the caulk tube extension 8 which provides a further illustration of the components of the invention relative to one another.

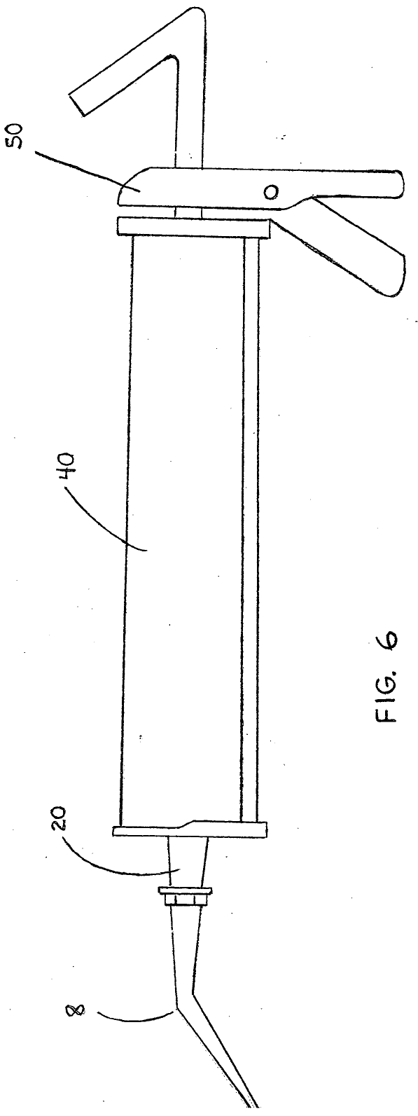

FIG. 5 shows how the caulk tube extension 8 interfits and mates with the caulk canister spout 20. The caulk tube extension 8 is manufactured from a harder material than is the caulk canister spout 20 so that the caulk canister spout 20 is self-threaded when the caulk tube extension 8 is mated thereto. Another advantage of this caulk tube extension 8 is that it can be quickly and easily attached to and removed from a standard caulk canister spout 20.

FIG. 6 illustrates the caulk tube extension 8 attached to the caulk canister spout 20 while the caulk canister 40 is positioned in a caulk gun 50. Thus, when caulking compound is passed through the caulk tube extension 8, it can be directionally applied to hard to reach surfaces.

It should be readily understood by those skilled I the art that the caulk tube extension 8 may be disposable or re-useable.

While the invention has been described in connection with a preferred embodiment, it will be understood that it is not intended that the invention be limited to that embodiment. On the contrary, it is intended to cover all alternatives, modifications and equivalents as may be included within the spirit and scope of the invention as disclosed.

As to the manner of usage and operation of the present invention, the same should be apparent from the above disclosure, and accordingly no further discussion relevant to the manner of usage and operation of the present invention shall be provided.

With respect to the above description then, it is to be realized that the optimum dimensional relationships for the parts of the present invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered illustrative of only the principles of the present invention. Further, since numerous modifications and changes will readily occur to those skilled in the art, it is not desired to limit the claims to the exact construction and operation shown and described, and accordingly, all suitable modifications and equivalents may be resorted to, falling within the scope of the claims.

1. A caulk tube extension comprising:
an elongated cylindrical tube constructed of a substantially rigid material and having an open attaching end and an open dispensing end, the open attaching end containing a means for removable attachment to the spout of a caulk canister for receiving caulking compound therefrom, and the tube containing a first segment and a second segment, where the first segment extends from the open attaching end, and the second segment is integrally attached to the first segment and extends to the open dispensing end, and the longitudinal axis of the first segment is oriented at an angle to the longitudinal axis of the second segment.

2. The caulk tube extension of claim 1, wherein the means for attachment is a plurality of male threads disposed circumferentially in the interior of the open attaching end, the threads being constructed from a relatively high density polyethylene, and the exterior of the open attaching end containing a hex nut integrally connected to and disposed around its circumference.

3. The caulk tube extension of claim 1, wherein the angle is 45 degrees.
